(19) United States
(12) Patent Application Publication
(10) Pub. No.: US 2023/0341377 A1
(43) Pub. Date: Oct. 26, 2023

(54) DISCONNECTION AGENTS

(71) Applicant: DC Europa Limited, Babraham Cambridge Cambridgeshire (GB)

(72) Inventor: John David GRAINGER, Babraham Cambridgeshire Cambridgeshire (GB)

(73) Assignee: DC Europa Limited, Babraham Cambridge Cambridgeshire (GB)

(21) Appl. No.: 18/026,983

(22) PCT Filed: Sep. 21, 2021
(86) PCT No.: PCT/GB2021/052449

§ 371 Date: Mar. 17, 2023

(30) Foreign Application Priority Data
Sep. 21, 2020 (GB) 2014831.8

(51) Int. Cl. G01N 33/50; A61K 45/06

(52) U.S. Cl. CPC G01N 33/5032; A61K 45/06; G01N 2458/30; G01N 2500/10

(57) ABSTRACT

The invention relates to the use of disconnection agents before, or together with, toxic agents for the treatment of solid tumours.

The invention relates to the use of agents that disconnect tumour cells, and specifically compounds that activate Protein Kinase C (PKC) enzymes, used before or together with toxic agents, to enable the successful treatment of solid tumours.

Tumours are typically divided into two major types: liquid tumours (where the malignant cells are typically circulating in the blood stream, usually arising from cells in the haemopoietic lineage) and solid tumours (where the tumour is located, at least initially, in a single location in a solid tissue type). Over the last three decades there has been considerable progress in the treatment of liquid tumours, with many types of liquid tumour that would once have rapidly been fatal now amenable in almost every case to successful treatment.

By contrast, while there has been progress in the treatment of solid tumours, in many cases the best available treatment only slows the progression of the tumour in the majority of patients, and such tumours once diagnosed are still usually fatal. In some cases, such as the brain cancer glioblastoma or pancreatic ductal adenocarcinoma or triple-negative breast cancer, best available care delays death by only a matter of months despite in many patients substantially shrinking the tumour only for the residual disease to return more aggressively with resistance to all available treatment options.

This pattern of “partial efficacy”, delaying rather than curing, the tumour has led to the development of ever more toxic treatment strategies, attempting to eradicate the entire tumour cell population. Most recently, that has included harnessing the patient's own immune system to attack the tumour — an approach called immuno-oncology. Although such approaches were initially very encouraging, with a significant number of cures achieved in liquid tumours that were otherwise difficult to treat, they have for the most part been disappointing in their ability to cure most solid tumour types (with the notable exception of skin and lung cancers, which seem to be particularly sensitive to immuno-oncology strategies).

For most solid tumours the treatment paradigm is largely unchanged since more than half a century, consisting of surgery, radiotherapy, and chemotherapy. There have been a number of suggestions as to why newer approaches have largely failed to cure the majority of solid tumours. For example, it has been noted that while newer agents have increased toxicity towards the cancer cell population, they also have increased toxicity for the patient's normal cells (in other words, the therapeutic index is not sufficiently wide to allow enough of the toxic agent to be administered to destroy the tumour entirely without also killing the patient).

Recently, we and others have noted a property of certain tumour cells whereby they form physical connections with each other, and with normal cells in the nearby tissue (called the stroma). These connections have variously been called tunneling nanotubes (TNTs) and tumour microtubules (TMs). While the functions of these connections are unknown, there have been reports of small molecules, proteins and even whole organelles (such as mitochondria and ribosomes) being exchanged between cells connected by TNTs/TMs at least in vitro.

We have further extended this work to establish a method, being one aspect of the present invention, to measure the exchange of small molecules between cells in vitro. Using this method we have demonstrated that the connections between the cells are functional, and allow the cells to exchange small molecules.

The ability to exchange small molecules between cells has particular relevance to the survival of cells subjected to attack, whether with radiation, chemotherapy or the immune system. Irrespective of the nature of the attack, cells follow a common pathway that allows them to die in a controlled manner (called Programmed Cell Death). Although there are a number of routes such a cell can follow, depending on the particular circumstances that led it to approach death (for example the intrinsic apoptosis pathway, the extrinsic apoptosis pathway, the ferroptosis pathway, the pyropotosis pathway and others), they all have in common an increase in intracellular calcium ion concentration as the final, irreversible trigger for death.

Calcium ion concentration in the cell is controlled by the balance of influx and efflux. Under normal circumstances, the cell pumps calcium out of the cell to maintain a “low calcium” environment (not least to prevent the formation of insoluble calcium phosphate since the cell uses nucleotide phosphates as its principal intracellular energy currency). However, if the cell membrane becomes damaged (either physically or as a result of immunological attack or cytotoxin) or if the cell deliberately activates calcium influx channel proteins in the membrane, then calcium ions rapidly pour in irreversibly activating the cell death program.

However, in circumstances where the cell has an extensive network of connections with neighbouring cells, the calcium ions flowing in will equilibrate not just within the volume of a single cell, but will be spread across a network of several or indeed many cells. A useful analogy is to consider the cell as a bucket. When the tap is opened and calcium flows in, if sufficient enters it will fill the bucket (trigger the death program). However, if the bucket has sufficient holes in it (connections to other cells) then no matter how fast the tap runs, insufficient water will accumulate to fill the bucket (the death program cannot be activated). We have termed this process “calcium capacitance”. The greater the network of the cells all connected in this way, the greater will be the calcium capacitance and the stronger the resistance to death.

In this context, connections between tumour cells and the normal, healthy cells of the stroma may be particularly important. If two identical tumour cells are connected, and both come under attack, then the presence of connections between them will be irrelevant (according to our analogy, linking two identical buckets under two separate, identical taps will not affect the rate at which they fill). By contrast, if a tumour cell coming under attack is linked to a normal cell that is resistant to that attack, the tumour cell will be less likely to reach the threshold calcium ion concentration required to trigger death (linking the bucket to another that has no tap pouring in water will halve the rate at which the original bucket fills). The more connections there are to a network of normal cells, the harder it will be for the tumour cell to die in response to any stimuli, no matter how toxic. Further, the more exposed tumour cells attacked by the immune system or a toxin form a network with less damaged cells at the tumour core to resist programmed cell death. Forming functional connections with neighbouring cells, and particularly with normal, healthy cells in the stroma, thereby provides a mechanism of resistance to death that is independent of the nature of the toxic insult. This “cloak of invincibility” conferred by networking can make the cell functionally impossible to kill.

While there is no benefit to being connected to another cell identical to you (in terms of protection from death in response to an insult that is toxic to both of you), nevertheless connections between tumour cells may also be protective because the tumour cell population is not homogeneous. One of the key characteristics of tumour cells is genomic instability, meaning that they accumulate mutations (including larger changes in chromosomal architecture) at a much faster rate than healthy cells. As a result, during tumour cell growth and proliferation, the daughter cells deviate from the parental cells, and may acquire different properties. For example, some cells may express high levels of an antigen targeted by a monoclonal antibody drug or cytotoxic T lymphocytes, while other cells express lower levels. Such a situation would allow the tumour cells to exploit these differences to protect themselves from the toxic attack of the antibody drug, in just the same way that connections with healthy cells can protect tumour cells. The cells with high antigen expression, who subsequently are triggered to die with an influx of calcium ions, can share that calcium load with neighbours who have low antigen expression (and so are not triggered for calcium ion influx), such that neither cell reaches the threshold calcium ion concentration required to die.

A third mechanism by which cellular connections can promote tumour survival is by allowing resistance to operate in trans, that is that resistance to a toxic insult, such as chemotherapy, in one cell can protect neighbouring cells to which it is connected. For example, if one cell expresses an enzyme that metabolises a cancer drug, then drug levels in all cells connected to that cell will be lowered by the activity of the enzyme in one cell—since the cancer drug will equilibrate between the cells that are connected. Similarly, if one cell expresses a pump that expels a cancer drug, then drug levels in all cells connected to that cell will be lowered by the action of the pump in one cell.

WO2016/044790A1 describes methods for treating brain metastasis. WO2015/188288A1 describes specific modulators of connexin hemichannels. WO02/063298A1 describes a gap junction permeability assay. WO2018/187647A1 describes methods and compositions for treatment of neurological diseases, disorders or conditions. WO2020/243359A1 describes compounds for use in anti-cancer immunotherapy. WO2006/096606A1 describes the use of PKC-activating compounds as cardioprotectants and as apoptosis-inducing anti-tumour agents. WO2009/129361A1 describes macrocyclic compounds and methods of making and using them. U.S. Pat. No. 5,770,593A describes a method of determining pharmaceutical composition preparations for use in anti-neoplastic therapy. WO2017/083783A 1 describes methods of cancer treatment. WO2017/156350A1 also describes methods of cancer treatment. El-Rayes et al. (2008; Pancreas 36: 346-352) relates to Protein Kinase C: a target for therapy in pancreatic cancer. Sidorova & Matesic (2008; Pharmaceutical Research 25: 1297-1308) describes the protective effect of chaetoglobosin K on lindane- and dieldrin-induced changes in astroglia and identification of activated signalling pathways.

A method according to one aspect of the present invention is to treat an individual who has a solid tumour with an agent that causes the disconnection of neighbouring cells. Such a treatment is not expected to have significant impact on the tumour cells, or the progression of the cancer, by itself. However, by initiating the disconnection, the agent removes the “cloak of invincibility” of the connected tumour cell networks, and thereby renders the entire tumour cell population susceptible to toxic insult. As a result, when the patient is treated with an appropriate agent toxic to the particular disconnected tumour cells, the patient will subsequently be cured (as opposed to the cancer simply being delayed by killing some, but not all, of the tumour cells) or experience substantial reduction in tumour burden by the killing of those disconnected cells that are susceptible to the toxic agent. A critical aspect of the present invention is therefore the need to treat the individual either sequentially (first with the disconnection agent and then with the toxic agent) or simultaneously or concomitantly (with the disconnection agent and the toxic agent at the same, or overlapping, times). It would not be a method according to the present invention to treat the individual with a solid tumour with the disconnection agent alone, or to treat them with a toxic agent followed by the disconnection agent. The sequence of treatment with the two types of agent is therefore a critical component of the invention.

According to the invention, there is provided a method of treating a mammal with a solid tumour in need of such treatment, comprising administration of a disconnection agent prior to, or concomitant with, administration of a toxic agent toxic to cells of the tumour.

According to the invention, there is provided a disconnection agent and a toxic agent for use in a method of treating a mammal with a solid tumour, wherein the disconnection agent is administered prior to, or concomitant with, the toxic agent.

According to the invention there is provided the use of a disconnection agent and a toxic agent in the manufacture of a medicament for a method of treating a solid tumour, wherein the disconnection agent is administered prior to, or concomitant with, the toxic agent.

The patient may be a human or animal. Preferably, the patient is a human.

The nature of the toxic agent to be used in sequence or combination with the disconnection agents according to the method of the present invention is not germane to the invention. Such agents are selected from those well known in the art to be toxic to the tumour cells from the individual undergoing treatment in accordance with the method of the invention. Such toxic agents include, but are not limited to, radiotherapy, chemotherapy, antibody drug conjugates, cell therapy and immunotherapy. For example, for the treatment of glioblastoma, the chemotherapeutic agent temozolomide may be used either alone or in combination with surgery and/or radiation. For example, for the treatment of pancreatic cancer the chemotherapeutic regimen known as FOLFIRINOX, a combination of fluorouracil, leucovorin, irinotecan, and oxaliplatin, may be used. Alternatively, a combination of gemcitabine plus paclitaxel may be used. For example, for the treatment of triple-negative breast cancer, the chemotherapeutic agent paclitaxel may be used, either alone or in combination with PARP inhibitors such as olaparib (in patients with BRCA mutations) or with antibodies against PD1/PDL1 (in patients with PD1 or PDL1 antigen positivity). The most appropriate toxic agent to be used for a particular individual, being treated in accordance with the method of the present invention, will be selected according to the current best practice described in the art.

The unique advantage of treating the patient in accordance with the method of the current invention is the increased probability of achieving remission or a complete cure, whereby the toxic agent already well known in the art, typically achieves only a very low rate of cure (with most patients treated in accordance with the best regimens known in the art still dying as a result of the cancer, although the period of time taken to die may be increased compared to the untreated case). Treating the patient in accordance with the method of the present invention will result in a materially increased proportion of patients who are completely cured (that is, have no detectable residual disease at the end of the treatment period).

Any agent that promotes the disconnection of nearby cells, without being directly toxic, is a disconnection agent useful in accordance with the method of the invention. A straightforward method for determining whether an agent is, or is not, a disconnection agent in accordance with the treatment method of the present invention is described herein and is provided as one aspect of the invention. One example is the determination of network disruption by in vitro assessment of its ability to inhibit small molecule sharing between neighbouring cells, imaged by fluorescent dyes, the small molecule sharing (SMS) assay.

In a further aspect of the invention, agents that modulate the activity of the enzyme Protein Kinase C are further provided as examples of disconnection agents useful in the treatment method of the invention. We have shown that Protein Kinase C modulators disrupt sharing between cells using the testing method for identifying disconnection agents in accordance with the method of the invention. According to this aspect of the invention, an individual with a solid tumour would be treated with an effective dose of a Protein Kinase C modulator (as the disconnection agent), and then with a suitable toxic agent selected from those well known in the art to be toxic for the tumour cells of the patient, either sequentially where the treatment with the Protein Kinase C modulator is performed before treatment with the toxic agent, or simultaneously whether the treatment with the Protein Kinase C modulator and the toxic agent occur at the same, or overlapping, times.

Examples of molecules that are PKC modulators that are disconnection agents for use in accordance with the treatment method of the invention include, but are not limited to, esters of phorbol such as 12-O-tetradecanoyl-phorbol-13-acetate (also known as PMA or TPA), N,N-dimethyl-3-aminomethylbutanoyl-phorbol-13-acetate and 12-butanoyl-phorbol-13-acetate, probol ester analogs such as indolactam V, (2E,4E)-N-[(2S,5S)-1,2,3,4,5,6-Hexahydro-5-(hydroxymethyl)-1-methyl-2-(1-methylethyl)-3-oxo-1,4-benzodiazocin-8-yl]-5-[4-(trifluoromethyl)phenyl]-2,4-pentadienamide (also known as TPPB) and bryostatin 1 and 2, and Indolactam V. For the avoidance of doubt, not all agents reported to modulate PKC isoenzymes may be disconnection agents in accordance with this aspect of the current invention. For example, as illustrated in Example 2, ingenol mebutate may be inactive in the simple cell-based assay of cellular network formation. It may be necessary to experimentally determine, for each such PKC modulator, whether they are a disconnection agent by performing a test as described herein.

A method to test whether an agent is a disconnection agent may comprise the following steps:
cells are placed into in vitro culture;
cells in two (or more) separate populations are labelled with two (or more) different dyes, at least one of which is trapped inside cells without reacting with the macromolecular components of the cell;
cells from the various labelled populations are mixed, and then incubated for a period during which connections may be formed between neighbouring cells.

During this period, some wells containing the mixture may optionally be exposed to a test agent;
at the end of the incubation period, the level of each dye in each cell is determined by a suitable method, such as flow cytometry.

An example of a suitable assay is provided in Example 1. Data obtained from the testing of many different agents against several different cancer cell lines is provided in Example 2. It will be obvious to those skilled in the art that such a test system is simple to establish, robust and reliable and does not require an inordinate amount of effort to perform. Further, it is evident from the output of the assay using several different cell lines that it is not possible for a person of ordinary skill in the art to predict the outcome from the testing, such that some agents that are structurally and functionally closely related (such as Bryostatin-1 and Bryostatin-2) differ substantially in their properties as a disconnection agent as determined empirically in this test system.

Another embodiment of testing the potential of TM disconnection properties of a PKC activator is the in vivo assessment of its ability to reduce the expression of GAP-43. A PKC modulator of interest may inhibit both small molecule sharing or the expression of GAP-43, Conexion-43 or TTY-H1.

For the avoidance of doubt the term Protein Kinase C includes all the isozymes of Protein Kinase C. For example, this includes, but is not limited to, the classical or conventional Protein Kinase C isoenzymes alpha, betal, beta2 and gamma, as well as the novel Protein Kinase C isoenzymes delta, epsilon, eta and theta, as well as the atypical Protein Kinase C isoenzymes iota/lambda and zeta. Preferably, the disconnection agent is a modulator of the classical or conventional Protein Kinase C isoforms, alpha, betal, beta2 and/or gamma. Preferably, the disconnection agent is a stimulator of the activity of the Protein Kinase C isoenzyme(s). It is possible that only a subset of PKC isozymes have to be activated for the disconnection of particular tumour networks. Examples of PKC modulators against PKC families and isozymes are shown in Example 3.

Similarly, the prior art on agents that modulate, and specifically activate, PKC isoforms strongly suggests that such agents would promote, rather than treat, cancer. For example, phorbol esters such as PMA and analogs such as TPPB have been referred to extensively in the public literature as tumour promoters, reflecting experiments in which PMA is painted onto the skin of animals and induces the formation of tumours. Consistent with this extensive literature, PMA purchased from a commercial supplier is accompanied by hazard warnings of the cancer-causing properties of the substance. Further PKC inhibitors are commercially researched at the time of this invention as anti-cancer agents by biopharmaceutical companies such as Ideay (https://www.ideayabio.com/pkc/). One with ordinary skill in the art would therefore conclude that attempting to treat cancer with agents that activate PKC would be unlikely to be beneficial, and indeed would very likely be detrimental. Set against this background, a method of the present invention, in which individuals with a solid tumour are treated with a PKC activator (followed by, or concomitant with, an agent toxic to the tumour cells) is surprising. The PKC activator may be broad or selective to particular isozymes.

There are, however, a few examples in the literature where agents that activate PKC enzymes have been employed to treat cancer, or have been ascribed properties which may be considered consistent with a possible beneficial effect in the treatment of cancer. For example, the compound ingenol mebutate has been approved for the treatment of the proliferative disease actinic keratosis, in which skin cells showed rapid and poorly controlled proliferation (while actinic keratosis is not defined as a solid tumour, it nevertheless has many properties in common with tumours and one might reasonably think that agents that treat actinic keratosis may have application in at least some solid tumours). However, it will be noted that a trivial extension of this observation, for example proposing that ingenol mebutate may be useful to treat a solid tumour in the same way it is used to treat actinic keratosis, would not be an example according to the present invention since it lacks at least two of the essential features of the present invention: (1) as shown in Example 2, ingenol mebutate, although it is a PKC activator, does not disconnect several different tumour cell types in the assay for disconnection provided as an aspect of the present invention to determine whether agents are disconnection agents for use in the method of treatment aspect of the invention; and (2) ingenol mebutate is used as a monotherapy to treat actinic keratosis, whereas an essential feature of the method of treatment of the present invention is the sequential or simultaneous use of a disconnection agent with a toxic agent. Nothing in the prior literature would therefore lead one of ordinary skill in the art to suppose that a particular sequential or combination treatment involving ingenol mebutate would be particularly effective for the treatment of solid tumours.

Similarly, it has been proposed that PKC activators could be used to treat certain tumours by directly inducing cell death (see WO2006/096606A1), although there is no demonstration that such a strategy is indeed effective in vivo. Importantly, however, such disclosure relates to the use of PKC activators as direct toxic agents for the treatment of cancer, and would not fall under the scope of the present invention because they do not teach the specific technical feature of combining the PKC activator (sequentially or concomitantly) with an agent toxic to the particular tumour type. This feature may be an important aspect of the present invention, since disconnection of the cells in the absence of a subsequent toxic insult has no effect on the tumour and may indeed be expected to be harmful to the normal tissues of the patient. Nothing in the prior literature would therefore lead one of ordinary skill in the art to suppose that a particular sequence or combination treatment involving PKC activators (even those that are disconnection agents, as defined in the present invention) would be particularly effective for the treatment of solid tumours.

The invention may comprise the following aspect: the use of a disconnection agent for treatment of solid tumours, where (a) the tumour is not glioblastoma and (b) the disconnection agent is used before a toxic agent.

Solid tumours treated in accordance with the present invention may not include cancers of the nervous system, such as brain tumours. For example, they may not include glioblastomas.

A critical component of the present invention is that the disconnection agent is administered to the patient in need of treatment before, or concomitant with, the administration of a toxic agent. The toxic agent is any agent known in the art to be toxic for the particular tumour affecting the patient undergoing treatment. Optionally, the patient may receive more than one dose of the toxic agent, or may receive more than one toxic agent, after the administration of the disconnection agent. The toxic agent includes, but is not limited to, chemotherapeutic agents, radiation therapy, immunotherapy or any combination thereof.

The disconnection agent must be administered in a time window prior to administering the toxic agent, such that the tumour cells are disconnected as a result of administration of the disconnection agent at the time when the toxic agent is subsequently administered. While the precise timing will depend on the kinetics of disconnection in response to the particular disconnection agent, this window will be a period of between 28 days and one minute; preferably between 14 days and one hour; more preferably between 7 days and one hour. In a preferred embodiment of the present invention the disconnection agent is administered approximately 24 hours prior to the toxic agent.

Optionally, where the toxic agent (or several toxic agents) are to be administered on more than one occasion, in accordance with the optimum use of the particular toxic agent(s) known in the art, then the disconnection agent may be administered prior to the first and each subsequent administration of the toxic agent(s). In a preferred embodiment of the present invention the disconnection agent is administered before the first administration of any toxic agent to the patient.

Optionally, more than one disconnection agent may be administered simultaneously, or effectively simultaneously, in order to achieve optimum disconnection of the tumour cells prior to the administration of the toxic agent(s).

The dose of the disconnection agent(s) to be used in accordance with the invention will be determined experimentally in accordance with methods well known in the art. The dose administered may be between 0.001 mg and 10 grams; preferably between 0.01 mg and 1 gram; more preferably between 0.1 mg and 500 mg. In a preferred embodiment of the present invention the dose of the disconnection agent will be between 5 mg and 500 mg. The dose of the disconnection agent may be 0.5 mg to 200 mg, such as 1 to 10 mg. Optionally, the dose of the disconnection agent may be adjusted according the characteristics of the particularly patient requiring treatment, for example according to the body weight, body mass index, body surface area, liver function or kidney function. In each case, the dose, route of administration and timing of administration are chosen so as to maximise the degree of disconnection of the tumour cells at the time of administration of the toxic agent(s).

Another aspect of the present invention, envisages the use of PKC modulators as disconnection agents in all solid tumours, including glioblastoma, where the PKC modulator is used before or concomitant with a toxic agent. The term PKC modulator includes, but is not limited to, the specific compounds that have been identified to have disconnection activity according to the present invention, including esters of phorbol such as 12-O-tetradecanoyl-phorbol-13-acetate (also known as PMA or TPA), N,N-dimethyl-3-aminomethylbutanoyl-phorbol-13-acetate and 12-butanoyl-phorbol-13-acetate, probol ester analogs such as indolactam V, (2E,4E)-N-[(2S,5S)-1,2,3,4,5,6-Hexahydro-5-(hydroxymethyl)-1-methyl-2-(1-methylethyl)-3-oxo-1,4-benzodiazocin-8-yl]-5-[4-(trifluoromethyl)phenyl]-2,4-pentadienamide (also known as TPPB) and bryostatin 1 and 2, and Indolactam V. More generally, the term PKC modulator applies to those agents identified as modulators of one or more PKC isoforms using assays for PKC activity well known in the art. Preferably, the PKC modulator is a stimulator of PKC activity (at least under the conditions, including dose and timing, that it is used to treat a patient in need of such treatment). More preferably, the PKC modulator has also been shown to be a disconnection agent according to the test provided in another aspect of the present invention.

A critical component of the present invention is that the PKC modulator is administered to the patient in need of treatment before or concomitant with the administration of a toxic agent. The toxic agent is any agent known in the art to be toxic for the particular tumour affecting the patient undergoing treatment. Optionally, the patient may receive more than one dose of the toxic agent, or may receive more than one toxic agent, after the administration of the PKC modulator. The toxic agent includes, but is not limited to, chemotherapeutic agents, radiation therapy, immunotherapy or any combination thereof.

The PKC modulator must be administered in a time window prior to administering the toxic agent, such that the tumour cells are disconnected as a result of administration of the PKC modulator at the time when the toxic agent is subsequently administered. While the precise timing will depend on the kinetics of change in PKC activity and subsequent disconnection of the tumour cells in response to the particular PKC modulator, this window will be a period of between 28 days and one minute; preferably between 14 days and one hour; more preferably between 7 days and one hour. In a preferred embodiment of the present invention the PKC modulator is administered approximately 24 hours prior to the toxic agent.

Optionally, where the toxic agent (or several toxic agents) are to be administered on more than one occasion, in accordance with the optimum use of the particular toxic agent(s) known in the art, then the PKC modulator may be administered prior to the first and each subsequent administration of the toxic agent(s). In a preferred embodiment of the present invention the PKC modulator is administered before the first administration of any toxic agent to the patient, or before or together with radiotherapy.

The dose of the PKC modulator to be used in accordance with this aspect of the invention will be determined experimentally in accordance with methods well known in the art. The dose administered will be between 0.001 mg and 10 grams; preferably between 0.01 mg and 1 gram; more preferably between 0.1 mg and 500 mg. In a preferred embodiment of the present invention the dose of the disconnection agent will be between 1 mg and 500 mg. Optionally, the dose of the PKC modulator may be adjusted according the characteristics of the particularly patient requiring treatment, for example according to the body weight, body mass index, body surface area, liver function or kidney function. In each case, the dose, route of administration and timing of administration are chosen so as to maximise the modulation of PKC activity and therefore the degree of disconnection of the tumour cells at the time of administration of the toxic agent(s).

In a preferred embodiment of this aspect of the invention the PKC modulator is TPPB, or an analog of TPPB, the solid tumour type is a glioblastoma and the toxic agent is radiotherapy and/or administration of temozolamide. Preferably the dose of the TPPB or TPPB analog is between 0.5 mg and 200 mg, such as 1-10 mg. Preferably the TPPB or TTPB analog is dosed 24 hours prior to the radiotherapy and/or temozolamide.

In another embodiment the PKC modulator is administered intermittently allowing for the disconnection of tumour networks before or during each cycle of toxic anti-cancer treatment. This aspect minimizes disconnection of networks between healthy cells while providing sufficient tumour cell disconnection to enhance the effect of a toxic agent.

In another embodiment, the PKC modulator is administered locally e.g., at a time of surgical removal. This embodiment may be particularly useful in the treatment of glioblastomas, to ensure sufficient amount of PKC activator enters the CNS. Further, the local administration may improve the therapeutic window of the PKC activator by avoiding the disconnection of healthy cell networks.

Another aspect of the present invention is a method for determining whether an agent is a disconnection agent.

According to the invention, there may be provided, a method to identify disconnection agents useful for the treatment of tumours comprising the following steps:
i. obtaining donor and acceptor cells, where the donor cells are distinguishable from the acceptor cells;
ii. loading the donor cells with an intracellular marker substance that can be transferred from the donor cells to the acceptor cells via connections;
iii. mixing the donor cells and the acceptor cells and culturing in the presence of a candidate disconnection agent, and optionally in the absence of a candidate disconnection agent;
iv. detecting the degree of transfer of the intracellular marker substance into the acceptor cells.

The method may further comprising the following steps:
v. identifying a disconnection agent which reduces or prevents transfer of the intracellular marker substance into the acceptor cells;
vi. producing a medicament consisting of or comprising the identified disconnection agent.

The invention may provide a disconnection agent produced by this method.

A test for disconnection agents in accordance with the present invention may have the following properties: a means to distinguish donor and acceptor cells and a marker substance that is transferred from the donor cells to the acceptor cells over a period of time, which can be accurately quantified. In order to perform the test, the donor cells may be loaded with a marker substance and then incubated in cell culture, such as an in vitro cell culture, with acceptor cells for a period during which the marker substance can be transferred if, and only if, the cells form connections between the donor and acceptor cells. After a period of time, the fraction of acceptor cells that now contain the marker substance, and are therefore inferred to have formed connections with the donor cells, is determined.

The method may comprise the step of labelling the donor cells and/or the acceptor cells, so that the donor cells are distinguishable from the acceptor cells. In a preferred embodiment of the test, the donor and acceptor cells are the same cell type, where donor and/or acceptor cells have been labelled with a dye that cannot be transferred to other cells prior to their use in the test, such that the donor and acceptor cells are labelled differently. Preferably the cells to be used are human cells. Preferably the cells to be used are derived from the tumour type to be treated using the disconnection agents identified in the test. Preferably the test is repeated using cells from more than one cell line or more than one donor. Preferably, the dye to be used to label the donor and/or acceptor cells forms a covalent link to macromolecular components of the cell, so that it is not readily transferred between the cells. Preferably, the dye to be used to label the donor and/or acceptor cells cannot diffuse out of the cells after labelling.

In a preferred embodiment of the test, the marker substance is a dye that (a) is unable to diffuse out of the cell once it has diffused in; and (b) does not interact with any component of the cell and is therefore free to diffuse between cells if they are appropriately connected. Preferably, the marker substance is a fluorescent dye so that it can be readily detected and quantified. Most preferably, the marker substance used in the test is Cell Tracker Green.

In order to perform the test, the donor and/or acceptor cells are labelled, and the donor cells are loaded with the marker substance, such that the marker substance can be readily detected in the donor cells, but not in the acceptor cells at the start of the test, using methods well known in the art. The donor and acceptor cells may be analysed by flow cytometry. In one example, the method may include simultaneous measurement of a different fluorescent dye marking each of the donor and acceptor cell populations and a third fluorescent dye representing the marker substance.

In the next step, the donor and acceptor cells may be incubated together in a cell culture well or flask for a period of time. Preferably the incubation is performed at 37° C. Preferably the incubation is performed under the conditions (such as the composition of the medium) that is normally used to grow the cells in cell culture.

Preferably donor and the acceptor cells are each added at a density of between 1×10³/ml and 1×10⁷/ml; more preferably at a density between 1×10⁴ and 1×10⁶/ml; most preferably at a density of approximately 1×10⁵/ml. Preferably the density of the donor and acceptor cells will be approximately the same, so that there is one acceptor cell for each donor cell. Preferably, the cells are plated into a cell culture well or flask with between 0.01 ml and lml per cm²; more preferably with between 0.066 ml and 0.33 ml per cm²; most preferably with 0.1 ml per cm². Preferably the cells are incubated together for a period between 30 minutes and 72 hours; more preferably between 2 hours and 48 hrs; most preferably between 2 hrs and 24 hrs.

In all cases, the conditions of the particular method used for the test should result in between 2% and 100% of the acceptor cells having detectable marker substance within them at the end of the incubation; more preferably between 10% and 100% of the acceptor cells will have detectable levels of maker substance at the end of the experiment; most preferably between 50% and 100% of the acceptor cells will have detectable levels of maker substance at the end of the experiment. Detectable levels of the marker substance is defined as a level more than 3 standard deviations above the average level detected in the acceptor cells prior to incubation with the donor cells.

The test may be performed in multiple identical replicate cell culture wells or flasks, such that different incubation conditions can be compared. Typically, several replicate wells or flasks are treated with the candidate disconnection agent added at some time after the donor and acceptor cells are mixed. Preferably the candidate disconnection agent is added between 1 second and 24 hours after the donor and acceptor cells are mixed; more preferably the candidate disconnection agent is added between 1 hour and 6 hours after the donor and acceptor cells are mixed; most preferably the candidate disconnection agents is added approximately 2 hours after the donor and acceptor cells are mixed.

The results obtained from donor cells and acceptor cells that have been cultured in the presence of a test candidate disconnection agent may be compared with the results obtained with the same donor cells and acceptor cells, but which have been cultured in the absence of a candidate disconnection agent or a known disconnection agent. This may provide a negative control. For example, at the end of the experiment the proportion of acceptor cells that have detectable levels of the marker substance may be compared with identical replicate wells or flasks to which the candidate disconnection agent was added and the identical replicate wells or flasks to which only the carrier or vehicle used to dissolve the candidate disconnection agent was added. Preferably, at least duplicate wells of each condition will be assessed; more preferably at least triplicate wells of each condition will be assessed. Most preferably, the conditions for the test will be chosen so that the coefficient of variation (the standard deviation between identical replicate wells or flasks divided by the mean of those replicates) is less than 10%.

The results obtained from donor cells and acceptor cells that have been cultured in the presence of a candidate disconnection agent may be compared with the results obtained with the same donor cells and acceptor cells, but which have been cultured in the presence of a known disconnection agent. This may provide a positive control.

Preferably, more than one dose of the candidate disconnection agent will be tested in different replicate wells or flasks. Preferably, a separate experiment will be performed in parallel to demonstrate that the candidate disconnection agent does not affect the viability of the cells used in the test under the conditions of the test (since agents that cause the cells to die, and in particular to burst open) risk generating a false-positive in the test.

Candidate disconnection agents that (a) reduce the proportion of acceptor cells that contain detectable levels of the marker substance at the end of a valid test that is (b) statistically significant using an appropriate statistical test with at least one dose level tested and (c) does not impact the viability of the cells used in the test under the conditions of the test is considered a disconnection agent in accordance with the method of the present invention.

A preferred embodiment of a method for identifying a disconnection agent according to the invention is the method of Example 1.

A method of identifying a disconnection agent according to the invention is relatively straightforward to perform using techniques well known in the art, and does not require undue experimentation to determine whether any given candidate disconnection agent is or is not a disconnection agent according to the present invention. Furthermore, the test demonstrably has considerable power to identify disconnection agents, since the vast majority (more than 99%) of random compounds are shown not to be disconnection agents when the test is applied; by contrast, agents that are known to cause disconnection by direct microscopic observation are essentially all correctly identified as disconnection agents by the present test. The test provided therefore meets the essential criteria for a useful test of having both very low false-positive and very low false-negative rates. It therefore provides a straightforward and reliable means by which individuals skilled in the art are able to conclusively determine both which agents are disconnection agents according to the present invention, and equally to determine which agents are not disconnection agents according to the present invention.

Some aspects of the present invention envisage agents to be administered as a treatment for patients requiring such treatment, as a pharmaceutical composition. The pharmaceutical compositions can be in the form of a solid, for example powders, granules, tablets, gelatin capsules, liposomes, polymeric nanoparticles, antibody drug conjugates, ROS-targeting pro-drugs or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax. Other appropriate pharmaceutically acceptable excipients and/or carriers will be known to those skilled in the art.

The pharmaceutical compositions according to the invention can also be presented in liquid form, for example, solutions, emulsions, suspensions or syrups. Appropriate liquid supports can be, for example, water, organic solvents such as glycerol or glycols, as well as their mixtures, in varying proportions, in water.

Solid tumours intended to be treated or cured by the method of treatment of the invention may include carcinomas, sarcomas, melanomas, and lymphomas. The tumour may be cancer of the bone or muscle, brain or nervous system, breast, endocrine system, eye, gastrointestinal tract, genitourinary tract, gynecologic tract, head, neck, skin, or respiratory tract. For example, the tumour may be breast cancer, pancreatic cancer, lung cancer, liver cancer, stomach cancer or ovarian cancer. The tumour may be a glioblastoma. The tumour is preferably a malignant, or cancerous, tumour.

According to the invention, further indications amenable to treatment include non-cancerous conditions.

The invention also provides a method of treatment, amelioration or cure of a solid tumour (including any symptomatic consequences of the presence of the tumour) by the administration to a patient of a sequence of compounds, compositions or medicaments as claimed herein.

Administration of a medicament according to the invention may be carried out by various routes including, but not limited to topical, oral, parenteral, intramuscular intravenous, pulmonary or subcutaneous. Administration may be systemic or local.

The administration dose envisaged for a medicament according to the invention may be between 0.1 mg and 10 g depending on the type of active compound used. According to the invention, there is provided a composition comprising a disconnection agent.

According to the invention, there is provided a composition comprising a disconnection agent and a toxic agent.

The compositions may be pharmaceutical compositions. The pharmaceutical compositions may comprise a pharmaceutically acceptable carrier, excipient or diluent.

According to the invention, there is provided a combined preparation comprising a disconnection agent and a toxic agent.

The composition or combined preparation may be for use as a medicament. For example, the composition or combined preparation may be for use in treating a solid tumour, as described herein.

### DEFINITIONS

The term “about” refers to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range. Hence, for example, the range of 4 to 20 carbon atoms specified in respect of (inter alia) formula I is intended to include all integers between 4 and 20 and all sub-ranges of each combination of upper and lower numbers, whether exemplified explicitly or not.

As used herein, the term “comprising” is to be read as meaning both comprising and consisting of. Consequently, where the invention relates to a “pharmaceutical composition comprising as active ingredient” a compound, this terminology is intended to cover both compositions in which other active ingredients may be present and also compositions which consist only of one active ingredient as defined.

As used herein, the term “disconnection agent” refers to an agent which reduces the ability of cells to exchange, share or transfer molecules with other cells, through connections, in particular through tumour microtubules. A disconnection agent may thus reduce the number of, inhibit the function of, or inhibit the formation of, tumour microtubules between cells. The microtubules may be between tumour cells, or between a tumour cell and a non-tumour cell. A disconnection agent thus includes an agent which, having been assessed according to a test disclosed herein, reduces the sharing of a marker substance (which may be measured as the proportion of acceptor cells positive for the donor dye by flow cytometric analysis). The reduction in sharing may be by at least 50%, preferably by at least 75% and more preferably by at least 90%, at a concentration that has no effect on cell viability (less than 10% of the cells dying in response to exposure to that concentration of the agent), using cells derived from one or more tumour types to be treated. For the avoidance of doubt, agents which have direct toxic effects (killing more than 10% of the cells at the concentration required to decrease sharing of intracellular dye) would not be “disconnection agents” in accordance with the present invention, at least for the tumour type where toxicity was observed.

As used herein, the term “toxic agent” is to be read as meaning an agent well known in the art to be toxic for cells derived from the tumour to be treated, either in vitro or in vivo. The term “agent” is intended to include exposure to radiation as well as treatment with cells (such as immune cells, whether normal cells or cells modified by genetic engineering, such as CAR-T cells), in addition to more conventional therapeutic agents such as biological proteins, peptides and chemicals. The particular “toxic agent” to be used when practicing the method of the present invention is not germane to the invention itself, and is selecting among radiotherapeutic, chemotherapeutic or immunotherapeutic regimens already used to treat the particular solid tumour (but which typically result in only partial treatment, whereby the progression of the cancer and the death of the patient are delayed rather than prevented).

As used herein, the term “combined preparation” refers to a “kit of parts” in the sense that the combination components (the disconnection agent and the toxic agent) as defined above can be dosed independently or by use of different fixed combinations with distinguished amounts of the combination components. The components can be administered simultaneously or concomitantly, or sequentially. The components of the combined preparation may be present in one combined unit dosage form, or as a first unit dosage form of one component and a separate, second unit dosage form of the other component. The ratio of the total amounts of the combination components to be administered in the combined preparation can be varied, for example in order to cope with the needs of a patient sub-population to be treated, or the needs of the single patient, which can be due, for example, to the particular disease, age, sex, or body weight of the patient.

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

The following examples are presented in order to illustrate the above procedures and should in no way be considered to limit the scope of the invention.

### FIGURES

FIG. 1 shows an example of an SMS assay according to the invention. In the figure, “A” denotes an unstained cell, “B” denotes a cell dye-stained cell, “C” denotes a cell tracker-stained cell, and “D” denotes a cell dye- and cell tracker-stained cell. In step 1, stain cells with a cell dye and plate. In step 2, stain cells with a cell tracker dye. In step 3, harvest cell dye-stained cells and pre-treat each set of stained cells separately with compounds of interest. In step 4, combine sets of stained cells and plate with compounds of interest. In step 5, harvest cells, stain with viability dye and assess cell dye transfer by flow cytometry. In the graph, the x-axis is CellTracker Fluorescence, the y-axis is Calcein AM Fluorescence, and the relative position of cell dye positive cells, cell tracker positive cells and cell dye plus cell tracker double positive cells depicted. Graphic shows dye can be transferred from a cell dye-stained cell to a cell tracker-stained cell but cell tracker cannot be transferred from a cell tracker-stained cell to a cell dye-stained cell;

FIGS. 2A-2C show examples of PKCm-mediated inhibition of cell dye-sharing in the SMS assay. FIG. 2A left graph shows SMS in TPPB-treated Hs578T cells and right graph shows SMS in bryostatin-treated Hs578T cells. FIG. 2B left graph shows SMS in PMA-treated Hs578T cells and right graph shows SMS in indolactam V-treated Hs578T cells. FIG. 2C left graph shows SMS in bryostatin 1-treated HepG2 cells and right graph shows SMS in bryostatin 2-treated U373 cells. In each graph, x-axis is concentration (μM) and y-axis is percentage of acceptor cells (%). In FIGS. 2A and B, upper horizontal dotted line is vehicle, lower horizontal dotted line is PMA (5 nM). In FIG. 2C, upper horizontal line is vehicle, lower horizontal dotted line is PMA (10 nM);

FIGS. 2D-2E show examples of PKCm not interfering with cell dye-sharing in the SMS assay. FIG. 2D left graph shows SMS in FR 236924-treated Hs578T cells and right graph shows SMS in 6-(N-decylamino)-4-hydroxymethylindole-treated Hs578T cells. FIG. 2E shows SMS in SC-9-treated Hs578T cells. In each graph, x-axis is concentration (i.t.M) and y-axis is percentage of acceptor cells (%); while the upper horizontal dotted line is vehicle and the lower horizontal dotted line is PMA (5 nM);

FIG. 3 shows in vivo MPLSM images of tumours treated with TPPB and radiotherapy. Top row is control, with images (left to right) at D0, D7, D14, D21 (D7 after RTx), D28 (D14 after RTx), D35 (D21 after RTx) and D42 (D28 after RTx). Middle row is TPPB continuous dosing day 0-28, with columns as top row (except no D42 image). Bottom row is TPPB 3 doses days 13, 14, 15, with columns as top row (except no D21 image);

FIG. 4 is a graph indicating differences between continuous and intermittent TPPB treatment. Depicted are: (i) vehicle; (ii) TMZ 25; (iii), TMZ 50, (iv) TPPB 0.7 2× per week+TMZ 25; and (v) TPPB 0.2 QD+TMZ 25. X-axis is days after tumour implantation and y-axis is relative tumour volume; and

FIG. 5 shows in vivo MPLSM images of tumours treated with TPPB and radiotherapy (column D) versus control treatment with radiotherapy alone (columns A, B and C) at various times (days 0, 7, 21, 28, 35, 42, 70 and 91 after initiation of radiotherapy in rows 1-8, respectively). Block E highlights treatment leading to absence of tumours. Two randomly selected fields of view are shown from each p animal, but the same field of view is depicted in each row (at each timepoint).

### EXAMPLES

### Example 1: A Test for Disconnection Agents (SMS Assay)

The SMS assay was developed to assess the transfer of cell dye (for example the acetoxymethyl [AM]-based Calcein AM), between cancer cells. The SMS assay can be applied to any cell lines forming tumour networks such as tumour microtubes (TM) or tunneling nanotubes (TNT), which enable the transfer of molecules across cells. The SMS assay can be used to assess networking capabilities of any cancer cell lines including HepG2 hepatocellular carcinoma cells, Hs578T cell line for human breast cancer, and U373 cell line for human glioblastoma.

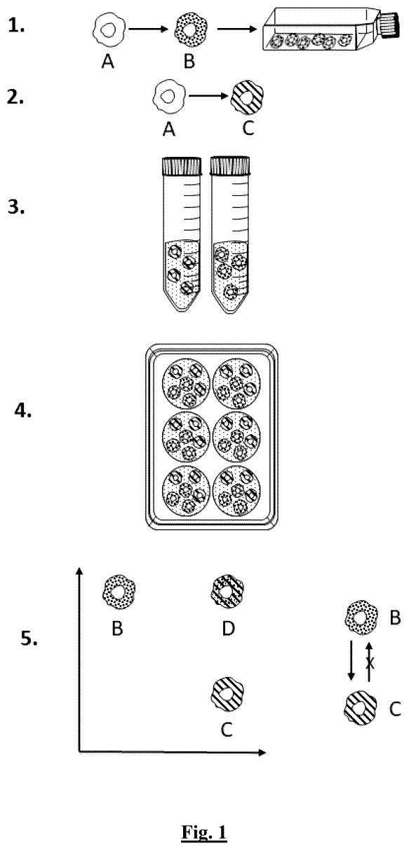

The SMS assay is illustrated in FIG. 1. For the SMS assay, two sets of cells are stained with the cell dye of choice and a customary cell tracker dye (for example cell tracker blue) using established staining techniques (steps 1 and 2). Each set of cells are then co-incubated with the compound of interest to assess its capabilities to modulate the cells small molecule sharing characteristics (step 3). Then the two sets of cells are combined on a plate, for example at a ratio of 1:1, and cultured in the presence of the compound of interest (Step 4).

Finally, live cells are evaluated for viability and analyzed by flow cytometry for the uptake of cell dye in the flow tracker-stained cells (Step 5). The comparison of cell tracker uptake against positive and negative control enables the assessment of the compound's ability to modulate the tumour networks including those formed by TM and TNTs

### Example 2: Demonstration of Disconnection of Tumour Cells in Vitro Using PKC Modulators

The SMS assay has been applied to assess the modulation of network sharing capabilities of Protein Kinase C Modulators (PKCm) at various concentrations in the Hs578 cell line for human breast cancer, U373 cell line of human glioblastoma, and HepG2 hepatocellular carcinoma cells. PBS vehicle was used as negative control and the PKCm PHORBOL 12-MYRISTATE 13-ACETATE (PMA) at a concentration of 5 nM was used as the positive control. Surprisingly, the application of the PKCm TPPB, PMA, Bryostatin 1, Bryostatin 2, and Indolactam V in the SMS assay showed a marked inhibition of cell dye-sharing at various doses that are not cytotoxic in one or more cell lines. However, the PKCm FR236924, SC-9, 6-(N-Decylamino)-4-Hydroxymethylindole, and Ingenol-3-angelate did not have any effect on cell dye-sharing at a concentration range of 0.1 to 30 μM.

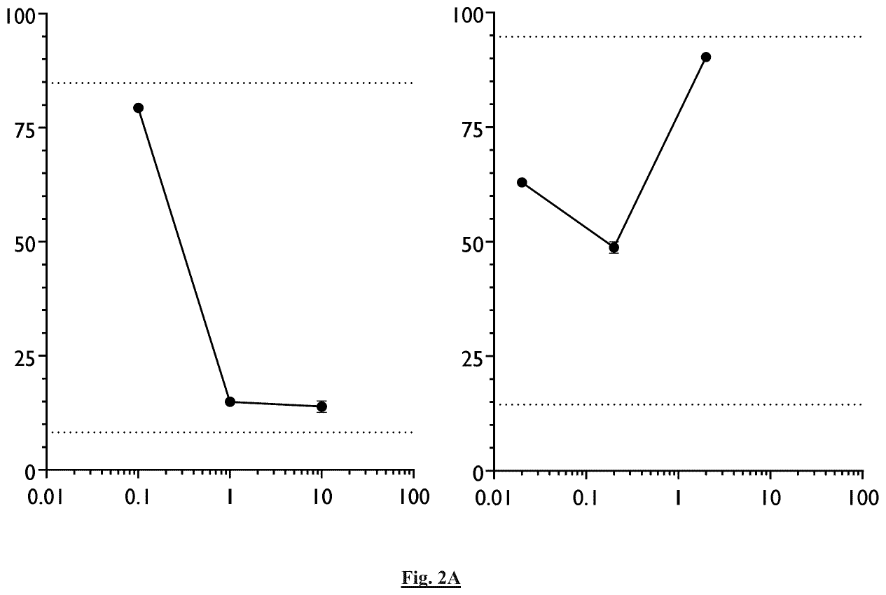

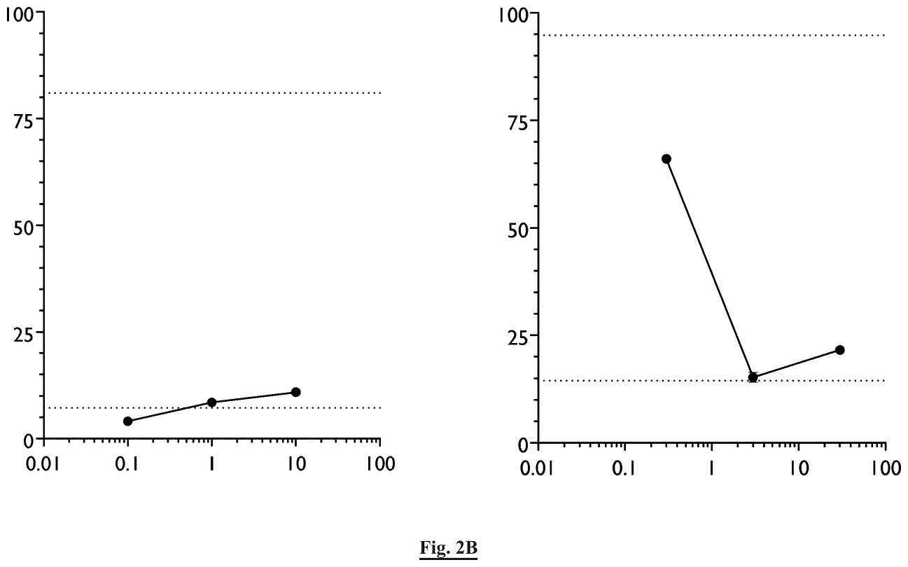

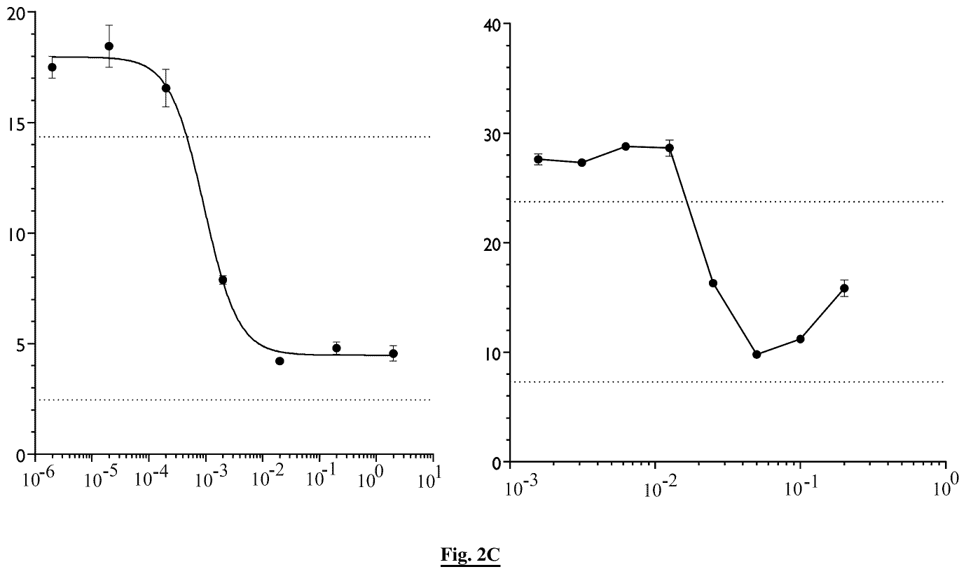

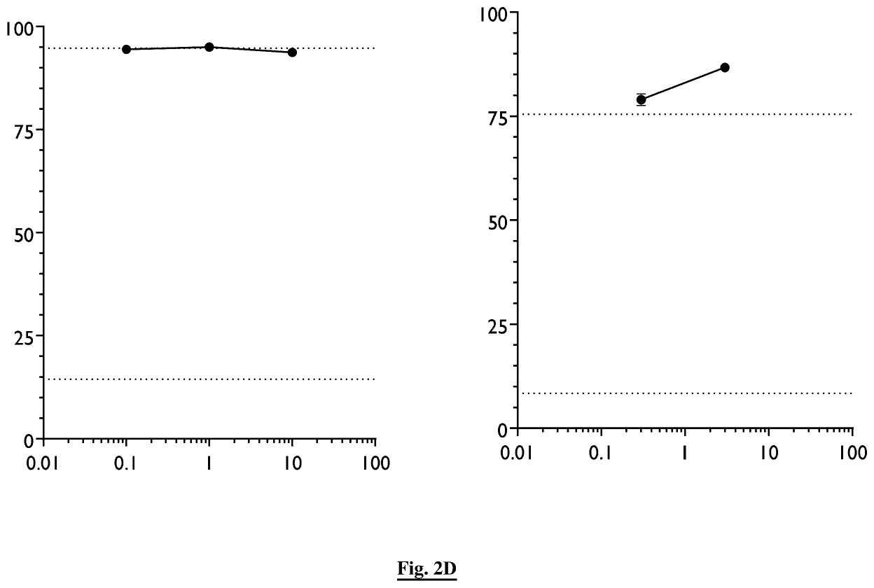

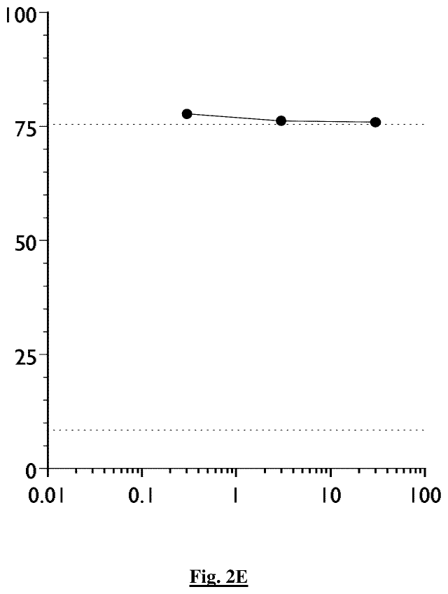

FIG. 2A and FIG. 2B show the percentage of cell tracker cells that were accepting the cell dye transfer from separately stained donor cells according to the SMS assay of Example. The percentage of acceptor cells detected is shown in comparison to vehicle (upper horizontal line) and PMA at 5-10 nM concentration (lower horizontal line). Active PKCm in the SMS assay (FIG. 2A) show a decrease in acceptor cells relative to vehicle at certain concentration. Non-active PKCm in the SMS assay (FIG. 2B) do not show a decrease of acceptor cells relative to vehicle. The upper limit of the tested concentrations was determined as the maximal non-cytotoxic dose.

The present invention thus identified TPPB, PMA, Bryostatin 1, Bryostatin 2, and Indolactam V as PKCm to disrupt tumour networks formed for example by TM and TNT. The invention extends to analogs of these compounds.

### Example 3: Specificity of PKC Modulators Against PKC Families and Isozymes

| Family | Enzyme | Gene | | TPPB | PMA | Bryostatin 1 | Indolactam V | Ingenol |
|---|---|---|---|---|---|---|---|---|
| cPKCs | PKCα | PRKCA | Alpha | ++ | + | + | + | + |
| | PKCβI/PKCβII | PRKCB | Beta | | + | + (βI) | ++ | + |
| | PKCγ | PRKCG | Gamma | | + | | + | + |
| nPKCs | PKCδ | PRKCD | Delta | + | + | + | ++ | + |
| | PKCε | PRKCE | Epsilon | + | + | + | + | + |
| | PKCη | PRKCH | Eta | | + | | + | |
| | PKCθ | PRKCQ | Theta | | + | | | |
| aPKCs | PKCζ | PRKCZ | Zeta | | | | | |
| | PKCι/λ | PRKCI | Iota/Lamda | | | | | |

+—activation,
++—strong activation,
blanks—no data
Protein Kinase C is a multifunctional protein kinase consisting of 9 isozymes that are clustered in 3 families of isozymes as depicted in the table above. PKCm are differentiated by their specificity to these PKC families and their isozymes. The differentiation of active and inactive PKCm in the SMS assay can be explained by differences in the activation of PKC families and individual isozymes. Such activated PKC families can be cPKC and/or nPKC. Such activated isozymes can be PKC alpha, beta, gamma, delta, epsilon, eta, and/or theta. The activity of a PKCm in the SMS assay depends on their potency against these targets, for example as noted below.
Alexander et. al. (2012) suggest TPPB and Bryostatin 1 activate PKC-alpha, delta and epsilon. Other PKCs not investigated.
Sharkey et al. (1984) demonstrated that DAG and PMA share a common binding site on PKC. This binding site (C1 domains; Giorgione et al. (2003)) on cPKCs and nPKCs is activated by PMA/DAG, however aPKC family isoforms have an atypical C1 domain that is activated by IP3 rather than DAG.
Wender et. al (2011) demonstrate activation and translocation of PKCβI following Bryostatin 1 treatment in transfected CHO cells.
No literature on the specificity of Bryostatin 2.
Masuda et. al. (2002) show binding of Indolactam V to synthetic peptide analogues of the stated PKC isozyme C1 domains.
Kedai et. al. (2004) provide inhibitor constants (Kᵢ) values for the binding of ingenol 3-angelate to the stated isozymes.
Parker et. al. (2008) performed KD of PKC-alpha with siRNA in U87MG cells, found no consistent impact on cell death in complete growth media, but did see an impact on cell cycle progression (stuck in S1 phase).
Shi et. Al. (2019) were able to develop a PD-L1^{KO}, EGFRVIII⁺ U373 cell line by CRISPR/Cas9.

### Example 4: Demonstration of Disconnection of Tumour Cells in Vivo Using TPPB and Radiotherapy in an Orthoptically Implanted Solid Brain Tumour

Tumour Implantation

8-10 weeks old male NMRI nude mice were used for all studies with human primary brain tumour cells. Cranial window implantation in mice was done in a modification of what has been previously described (see References), including a custom-made titanium ring for painless head fixation during imaging. 2-3 weeks after cranial window implantation, 30,000 tumour cells of the patient-derived S24 glioblastoma model were stereotactically injected into the mouse brain at a depth of 500 μm. Tumours were injected on day minus 35, i.e., 35 days before treatment start. Treatment was started on day 0.

Treatment

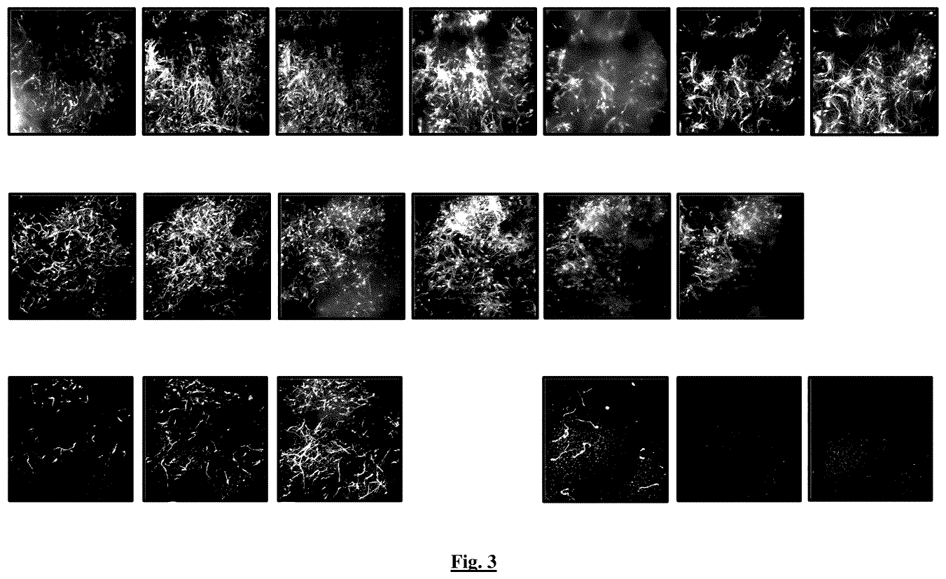

Control animals (top row of FIG. 3) were untreated. TPPB continuous treatment (middle row) was administered intraperitoneally once per day on days 0 to 28 at variable daily doses ranging from 10 to 700 μg/kg. TPPB 3 doses (bottom row of FIG. 3) were administered intraperitoneally once per day on days 13, 14 and 15 at 200 μg/kg.

Radiation Treatment (RTx)

Tumours were irradiated with 7 Gy on three consecutive days (days 14, 15, 16, total dose 21 Gy) in regions matching in tumour cell density using a 6 MV linear accelerator with a 6 mm collimator (adjusted to the window size) at a dose rate of 3 Gy min⁻¹ (Faxitron MultiRad225). The used radiation schedule is in the range of the commonly prescribed 60 Gy in 2 Gy. fractions for malignant glioma patients, assuming an α/β of ˜10 in the linear quadratic model and taking into account the radiation time of 3 days.

In Vivo Multiphoton Laser Scanning Microscopy (MPLSM) and Image Processing

MPLSM imaging was done with a Zeiss 7MP microscope (Zeiss) equipped with a Coherent Chameleon Ultrall laser (Coherent). MPLSM images were acquired by the ZEISS ZEN Software.

White to light gray areas of images in FIG. 3 depict S24 glioblastoma cells growing in the mouse brain. Black or dark gray areas depict vessels or background.

Results

Control treatment (top row) showed progressing S24 glioblastoma tumours in the mouse brain between days 0 and d7. Control animals had stable tumour burden between days 7 and 14, i.e., before radiotherapy (RTx), and stable tumour burden from days 28 to 42, i.e., after RTx on day 14. Tumour networks were identified as from day 7 and persisted through day 42, noticeable by the filament-like connections between the tumour cells.

TPPB continuous dosing (middle row of FIG. 3) showed no effect against control as described above. The tumour burden and connectivity between days 0 and 35 was similar to control. No images were taken on day 42.

Mice treated with 3 doses of TPPB on days 13, 14, and 15 had a similar tumour growth trajectory as untreated control animals on the pre-treatment days 0 to 14. Following treatment and RTx, mice treated with 3 doses of TPPB showed marked tumour regression on day 28 or 14 days after start of RTx. The tumour connectivity was markedly reduced on day 28. On days 35 and 42, i.e., 21 and 28 days after RTx, the S24 glioblastoma cells were barely noticeable, rounded, and completely disconnected amounting to complete remission from the tumour burden. No images were taken on day 21.

References:

Osswald, M.; Jung, E.; Sahm, F.; Solecki, G.; Venkataramani, V.; Blaes, J.; Weil, S.; Horstmann, H.; Wiestler, B.; Syed, M.; et al. Brain tumour cells interconnect to a functional and resistant network. Nature 2015, 528, 93-98.

### Example 5: Demonstration of the Impact of Disconnection Using TPPB Followed by Chemotoxic Insult on a Solid Tumour in Vivo

Tumour Implantation

NMRI nude mice were subcutaneously implanted with patient-derived glioblastoma cells from the Glio10535 model (Orthmann et al.) on day 0.

Treatment

Animals were stratified into the treatment groups on day 6 after tumour implantation to achieve similar average tumour volumes per group. Minimal tumour sizes were 44 mm³ and maximal tumour sizes were 247 mm³. Vehicle animals (n=9) were treated with a mix of DMSO, Tween20 and PBS. Each 5 animals were orally dosed with temozolomide chemotherapy at 25 mg/kg (TMZ 25) and at 50 mg/kg (TMZ 50) on consecutive days per week. Each week represents one chemotherapy cycle of 5 treatment days and 2 off-treatment days. TPPB was dosed intraperitoneally in combination with 25 mg/kg oral temozolomide either continuously at 0.2 mg/kg (n=10) or intermittently (n=10) at 0.1, 0.4, and 0.7 mg/kg starting with the last dose of every TMZ 25 cycle and ending the day before the next treatment cycle. Before the start of chemotherapy, TPPB was titrated to its target dose.

Results

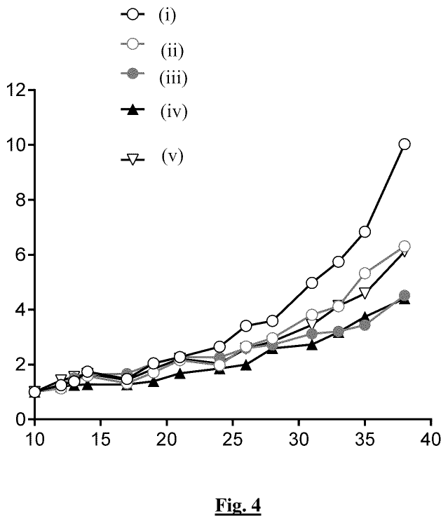

FIG. 4 shows the evolution of tumour volumes relative to treatment start on day 10 (relative tumour volume, RTV). By 38, when the majority of vehicle-treated animals reached critical tumour sizes, the anti-tumour effect achieved with intermittent TPPB dosing (TPPB 0.7 2× per week+TMZ 25 group) was additive to the effect of temozolomide 25 mg/kg monotherapy (TMZ 25), superior to continuous treatment with TPPB (TPPB 0.2 QD+TMZ 25 group), and similar to temozolomide 50 mg/kg monotherapy (TMZ 50).

The study confirmed that continuous dosing with TPPB has no effect over control, while intermittent dosing results in tumour growth inhibition.

References:

A. Orthmann , A. Hoffmann , R. Zeisig , J. Hayback, A. Jödicke, S. Kuhn, M. Linnebacher, J. Hoffmann, I. Fichtner, Therapeutic response to chemotherapeutic drugs of glioma-PDX and correlation to common mutations identified by panel sequencing, poster presentation (see https://www.epo-berlin.com/dokumente/2016_Poster_PAMM_Orthmann.pdf)

### Example 6: Demonstration of Complete Cure of an Orthoptically Implanted Solid Brain Tumour in Mice Using a Disconnection Agent Followed by Radiotherapy in Accordance with the Method of the Invention

Method

Orthotopic brain tumours were induced in NMRI mice using human patient-derived S24 glioblastoma cells exactly as described in Example 4. Once significant tumours were established in vivo (approximately 35 days after implantation), as shown by intravital microscopy exactly as described in Example 4, mice received 4 intraperitoneal injections on day -1, day 6 and day 13 and day 20, with Control mice receiving vehicle only and Treated mice receiving TPPB at 200 μg/kg. All mice then received radiotherapy on days 0, 1 and 2 exactly as described in Example 4, except that the radiation dose was 6 Gy as opposed to 7 Gy on each occasion (18 Gy total radiation dose as opposed to 21 Gy total radiation dose). The impact of treatment on the tumour was then monitored by intravital microscopy at intervals after the initiation of radiotherapy on day 0.

Results

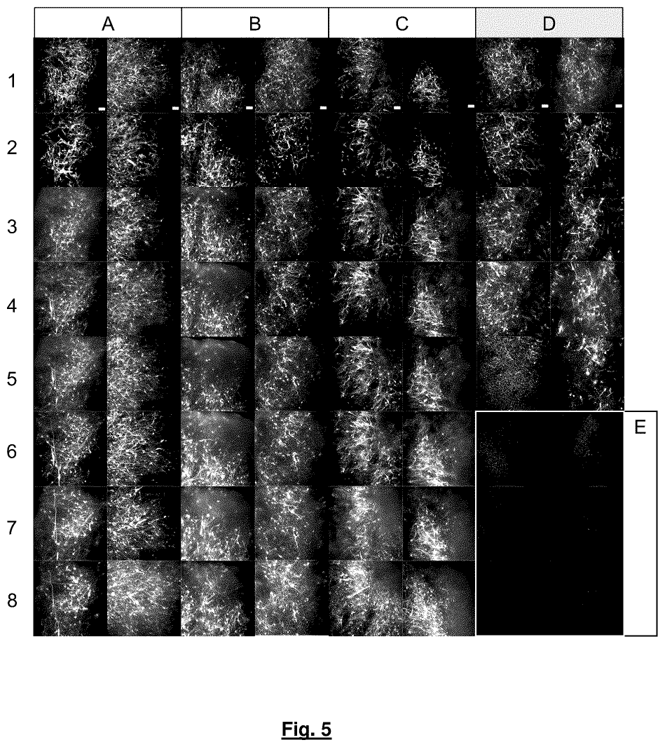

The results are shown in FIG. 5.

Mice that received control (vehicle) injections (columns A, B and C in FIG. 5), and were therefore not treated with a disconnection agent prior to radiotherapy in accordance with on aspect of the present invention, showed tumour progression throughout the study. Although there was an initial reduction in tumour cell number (white areas in each field of view in FIG. 5) immediately following radiotherapy, consistent with the toxic effect of the radiation, tumour burden subsequently increased throughout the observation period such that by day 91 after initiation of radiotherapy (row 8 in FIG. 5), the number of tumour cells had increased significantly in all fields of view from all animals. The progression observed in this study is consistent with reported studies using the same model (see references under Example 4), where progression is always seen following radiotherapy.

In marked contrast, mice that received active treatment with a disconnection agent (TPPB) initiated prior to radiotherapy, in accordance with one aspect of the present invention (column D in FIG. 5) showed a complete cure of the orthotopic solid brain tumour by day 91 after initiation of radiotherapy (row 8 in FIG. 5). By this time point, there were no detectable tumour cells (white areas in each field of view in FIG. 5) in any field of view. The only difference in treatment between the two groups was the use of a disconnection agent initiated prior to radiotherapy in the treated group (column D) compared to the control group (columns A,B and C) exactly in accordance with the method of the invention. A similar cure was observed among animals treated according to the method of the invention in Example 4. However, no other treatments (unrelated to the present invention) described in the literature have ever effected a cure in this model.

The impact of initiating treatment with a disconnection agent prior to radiotherapy was evident from day 35 after initiation of radiotherapy (row 5 in FIG. 5), when the tumour cells exhibited a marked change in morphology (in addition to the disconnection we observed, exactly as observed in Example 4), with a bright, punctate staining pattern visible at high magnification. It is likely that this represents the induction of apoptosis within the tumour cell population, which result in the complete absence of tumour cells observed at later timepoints (rows 6-8 in FIG. 5; highlighted as block “E”).

Treatment with a disconnection agent (TPPB) followed by radiotherapy, in accordance with the method of the invention, was well tolerated in all mice, with no behavioural or clinical signs associated with the treatment, and the mice remained well throughout the period of the study, in marked contrast to control mice that did not receive prior treatment with the disconnection agent, which increasingly demonstrated neurological symptoms consistent with the expanding tumour.

1. A method to identify a disconnection agent useful for the treatment of tumours, comprising the following steps:
i. obtaining donor and acceptor cells, where the donor cells are distinguishable from the acceptor cells;
ii. loading the donor cells with an intracellular marker substance that can be transferred from the donor cells to the acceptor cells via connections;
iii. mixing the donor cells and the acceptor cells and culturing in the presence of a candidate disconnection agent, and optionally in the absence of a candidate disconnection agent; and
iv. detecting the degree of transfer of the intracellular marker substance into the acceptor cells.

2. The method of claim 1, where the donor and acceptor cells are the same cell type.

3. The method of claim 2, where the donor and acceptor cells are non-tumour human cells.

4. The method of claim 3, where the cells are HepG2 cells.

5. The method of claim 2, where the cells are human tumour cells from the tumour type to be treated.

6. The method claim 5 where, the cells are glioblastoma-derived cells.

7. The method of claim 6, where the cells are U737 cells.

8. The method of any preceding claim, where the intracellular marker substance is Cell Tracker Green.

9. The method of any preceding claim, comprising labelling donor and/or acceptor cells, to obtain the donor cells and the acceptor cells which are distinguishable.

10. The method of any preceding claim, where the candidate disconnection agents is contacted with the donor and acceptor cells 1 to 6 hours after the donor and acceptor cells are mixed.

11. A method of treating a mammal with a tumour in need of such treatment comprising administration of a disconnection agent prior to, or concomitant with, administration of a toxic agent toxic to cells of the tumour.

12. The method of claim 11, where the disconnection agent is a modulator of Protein Kinase C activity.

13. The method of claim 12, where the disconnection agent is a stimulator of Protein Kinase C activity.

14. The method of claim 13, where the stimulator of Protein Kinase C activity is selected from: esters of phorbol (including PMA), bryostatin 1, bryostatin 2 and TPPB.

15. The method of any of claims 11-14, where the tumour type is not glioblastoma.

16. The method of any of claims 11-14, where the tumour type is glioblastoma.

17. The method of any of claims 11-14, where the tumour type is breast cancer, pancreatic cancer, lung cancer, liver cancer, stomach cancer or ovarian cancer.

18. The method of any of claims 11-17, where the disconnection agent is administered systemically.

19. The method of any of claims 11-17, where the disconnection agent is administered locally to the tumour.

20. The method of any of claims 11-19, where the disconnection agent is administered between 7 days and 1 hour prior to the administration of the toxic agent.

21. The method of any of claims 11-20, where the toxic agent is radiotherapy.

22. The method of any of claims 12-20, where the toxic agent is a chemotherapeutic agent.

23. The method of any of claims 11-20, where the toxic agent is an immunotherapeutic agent.

24. The method of claim 16, or any claim dependent on claim 16, where the disconnection agent is a stimulator of Protein Kinase C activity and the disconnection agent is administered between 7 days and 1 hour prior to the toxic agent.

25. The method of claim 24, where the stimulator of Protein Kinase C activity is selected from among the following list: esters of phorbol (including PMA), bryostatin 1, bryostatin 2 and TPPB.

26. A disconnection agent and a toxic agent for use in a method of treating a mammal with a tumour in need of such treatment, wherein the disconnection agent is administered prior to, or concomitant with, the toxic agent.

27. Use of a disconnection agent and a toxic agent in the manufacture of a medicament for a method of treating a mammal with a tumour in need of such treatment, wherein the disconnection agent is administered prior to, or concomitant with, the toxic agent.

28. A composition comprising a disconnection agent useful for the treatment of tumours, optionally wherein the composition is a pharmaceutical composition further comprising a pharmaceutically acceptable carrier, excipient or diluent.

29. A composition according to claim 28, comprising a toxic agent.

30. A combined preparation comprising a disconnection agent and a toxic agent.

31. A composition or combined preparation according to any of claims 28-30 where the disconnection agent is selected from among the following list: esters of phorbol (including PMA), bryostatin 1, bryostatin 2 and TPPB.

32. A composition or combined preparation according to any of claims 28-31, for use as a medicament.

33. A composition or combined preparation according to any of claims 28 to 31, for use in treating tumours.

34. The method of any of claims 1-10 further comprising the following steps:
v. Identifying a disconnection agent which reduces or prevents transfer of the intracellular marker substance into the acceptor cells;
vi. Producing a medicament consisting of or comprising the identified disconnection agent.

35. A disconnection agent produced by the method of claim 34.
